(19) United States
(12) Patent Application Publication
(10) Pub. No.: US 2021/0007343 A1
(43) Pub. Date: Jan. 14, 2021

(54) TICK AND TICK HEAD REMOVER

(71) Applicant: Paul Adams, Monroe, CT (US)

(72) Inventor: Paul Adams, Monroe, CT (US)

(21) Appl. No.: 16/925,382

(22) Filed: Jul. 10, 2020

Related U.S. Application Data
(60) Provisional application No. 62/873,567, filed on Jul. 12, 2019.

(51) Int. Cl. A01M 3/00; B65D 75/30; B65D 75/52

(52) U.S. Cl. CPC A01M 3/00; A01M 2200/011; B65D 75/52; B65D 75/30

(57) ABSTRACT

A tick remover device includes a substantially flat body defining a substantially v-shaped notch on one surface; a sharp point on another surface; and packaging material enclosing the flat body. A tick remover device can also include a substantially flat body defining a substantially v-shaped notch on one surface or edge; a sharp point on an another surface; and a protective member covering the sharp point to protect a user from the sharp point during use of the v-shaped notch. A packaged tick remover device as well as a method for using the packaged tick remover device are also disclosed.

### CROSS-REFERENCE TO RELATED APPLICATION

Benefit is claimed of U.S. Patent application No. 62/873,567, filed Jul. 12, 2019, and entitled “TICK AND TICK HEAD REMOVER”, the disclosure of which is incorporated by reference herein in its entirety as if set forth at length.

### BACKGROUND

The subject matter disclosed herein relates to a tick remover, and in particular a tick remover tool that is portable, sterile and quickly removes the tick from skin of animals or humans and can remove the tick head or barb when it separates from the tick body and is still embedded in the skin.

The method for removing a tick according to the prior art is well known. Tweezers are typically recommended to remove the tick but precision is required to remove a tick completely without leaving the head of the tick embedded in the skin as the tweezer needs to be compressed, just below the head of the tick (close to the skin) to remove the tick completely. The disadvantage of tweezers is that they cannot always remove the head of the tick if it is still embedded in the skin, and they are not easy to transport as they are long and need to be protected such as not to be damaged in transport.

There are tick remover products such as those disclosed in U.S. Pat. No. 5,447,511 and US2011/0098723. These products can be used to remove a tick but they cannot remove the tick head when needed, are not sterile and are larger than preferred for transport.

Ticks are typically found outside where people and their pets are hiking, hunting and camping where portability and weight are at a premium. People do not want to carry tweezers or larger tick remover tools in case they get a tick. If the tick head is embedded in the skin, it is recommended to remove it properly and promptly.

In addition, there are recommendations to save the tick for testing to determine if the tick is carrying any diseases such as Lyme or Rocky Mountain Spotted Fever. Sources recommend saving the tick that bit you in a sealed container and recording the date of the bite. Bringing the tick to a medical appointment may help the doctor in assessment of any possible illness.

There is a need for a sterile tool to facilitate tick removal and to minimize the risk of infection from either leaving the head in place or attempting to remove the tick head with a sharp implement such as a needle.

As a result, there is a need for a portable and sterile tool that can remove the tick and tick head especially when not near a medical facility or doctor.

### SUMMARY OF THE INVENTION

In accordance with the present disclosure, the foregoing issues are addressed and a tick remover is provided which is safe and effective in use to remove a tick, and also provides useful tools to remove a tick head or other objects from the skin when needed such as thorns, prickers, splinters, etc. The tool is portable and can be packaged sterile. According to the disclosure, a tick remover device is provided comprising a substantially flat body defining a substantially v-shaped notch on one surface; a sharp point on an another surface; and packaging material enclosing the flat body.

According to another aspect of the disclosure, a tick remover device is provided comprising a substantially flat body defining a substantially v-shaped notch on one surface or edge; a sharp point on another surface; and a protective means covering the sharp point to protect a user from the sharp point during use of the v-shaped notch to remove a tick.

According to still another aspect of the disclosure, a packaged tick remover is provided, comprising a package containing the tick remover device described herein, wherein the tick remover device is sterilized.

In still another aspect of the disclosure, a packaged tick remover is provided, comprising a package containing the tick remover device as described herein, wherein the package is made of paper.

In yet another aspect of the disclosure, a packaged tick remover is provided, comprising a package containing the tick remover device as disclosed herein, wherein at least one interior surface of the package has adhesive to capture the tick and/or hold the package in place while covering the sharp point.

In one embodiment, at least on one surface the package has adhesive to contain the tick within the packaging.

In another embodiment, the packaging further comprises a fastener to secure a tick in the package.

In a further embodiment, the package is free of adhesive.

In still another embodiment, the substantially flat body is a substantially flat metal plate.

In still a further embodiment, the sharp point is a needle attached to the substantially flat body.

In another embodiment, the sharp point is a needle formed integrally with the body.

In a further embodiment, the protective means comprises a breakaway shield which can be removed from the substantially flat body to expose the sharp point.

In still another embodiment, the packaging material is the protective means.

In a still further embodiment, the notch has an angle of between 2 and 60 degrees, and in another embodiment this angle is between 2 and 20 degrees.

In another embodiment, the v-shaped notch is defined by inner edges of arms of the plate member, and at least a portion of the inner edges are parallel.

In a further embodiment, the notch can have an opening width x of width between 1 and 10 mm and a length y between 2 and 12 mm.

In still another embodiment, the width of the slot is between 1 and 5 mm and the length is between 5 and 10 mm.

In a still further embodiment, the tick remover device has a width of between 0.25 and 2.5 cm.

In another embodiment, the tick remover device has a width of between 0.5 and 4 cm.

In a further embodiment, the packaged tick remover has a width between 0.5 and 5 cm.

In still another embodiment, the packaged tick remover has a width of less than 5 cm.

In a still further embodiment, the tick remover device has a length of between 1 and 8 cm.

In another embodiment, the tick remover device has a length between 2 and 6 cm.

In a further embodiment, the tick remover device has a length of less than 5 cm.

In still another embodiment, the packaged tick remover has a length of between 3 and 9 cm.

In a still further embodiment, the packaged tick remover has a length less than 9 cm.

In another embodiment, the tick remover device has a thickness of less than 2 mm.

In a further embodiment, the tick remover device has a thickness is less than 0.5 mm.

In still another embodiment, the packaged tick remover has a thickness of between 1 and 5 mm.

In a still further embodiment, the packaged tick remover has a thickness of less than 5 mm.

In another embodiment, the packaged tick remover has a thickness of less than 1.5 mm.

In a further embodiment, the packaged tick remover has a weight of less than 50 g.

In still another embodiment, the packaged tick remover has a weight less than 5 g.

In a still further embodiment, the packaged tick remover has a weight less than 2 g.

In another embodiment, the tick remover device has a weight of less than 10 g.

In a further embodiment, the tick remover device has a weight less than 2 g.

In still another embodiment, the tick remover device has a weight less than 0.5 g.

In a still further embodiment, the tick remover device has a volume less than 5.0 cm³.

In another embodiment, the tick remover device has a volume less than 0.5 cm³.

In a further embodiment, the tick remover device has a volume less than 0.1 cm³.

In still another embodiment, the packaged tick remover has a volume of less than 60 cm³.

In a still further embodiment, the packaged tick remover has a volume of less than 20 cm³.

In another embodiment, the packaged tick remover has a volume of less than 2 cm³.

In a further embodiment, sides of the V-shaped notch are beveled.

In still another embodiment, the substantially flat body comprises arms which define the V-shaped notch, and the arms are co-planar with the substantially flat body.

In a still further embodiment, the substantially flat body comprises arms which define the V-shaped notch, and the arms are bent at an angle greater than 0 and less than 90 degrees with respect to the body.

In another embodiment, a stiffening material is positioned along the substantially flat body.

In a further embodiment, the stiffening material comprises at least one rib formed on the body.

In still another embodiment, the packaging material covers at least the sharp point.

In a still further embodiment, the packaging material covers both sides of the tick remover device, and a portion of the packaging material can be removed away from a notch end of the tick remover device to expose the notch end for use, while remaining in place over the sharp point.

In another embodiment, a method for removing a tick from the skin is provided, the method comprising the steps of: removing a tick remover device from a packaging enclosing the tick remover device, wherein the tick remover device comprises a substantially flat body defining a substantially v-shaped notch on one surface; a sharp point on another surface; and packaging material enclosing the flat body; placing the notch against a tick embedded in the skin to remove the tick; using the sharp point to remove any remaining portions of the tick; and sealing the removed tick into the packaging.

### BRIEF DESCRIPTION OF THE DRAWINGS

A detailed description of the present disclosure follows with reference to the attached drawings, wherein:

FIG. 1A is a schematic illustration of a tick remover in accordance with a non-limiting embodiment;

FIG. 1B is a side view of the tick remover as shown in FIG. 1A in accordance with an embodiment having a flat surface;

FIG. 1C is a side view of the tick remover in FIG. 1A accordance with an embodiment with an angled or radius surface;

FIG. 2A is a schematic illustration of a tick remover in accordance with another non-limiting embodiment;

FIG. 2B is a cross-sectional side view of the tick remover shown in FIG. 2A;

FIG. 2C is a schematic illustration of a tick remover in accordance with another non-limiting embodiment;

FIG. 2D is a cross-sectional side view of the tick remover shown in FIG. 2C;

FIG. 2E is a schematic illustration of a tick remover in accordance with another non-limiting embodiment;

FIG. 2F is a side view of the tick remover shown in FIG. 2E;

FIG. 2G illustrates an alternative configuration of a tick remover in accordance with a non-limiting embodiment;

FIG. 3A is a schematic illustration of a tick remover in accordance with another non-limiting embodiment;

FIG. 3B is a side view of the tick remover shown in FIG. 3A;

FIG. 4A is a schematic illustration of a tick remover in accordance with another non-limiting embodiment;

FIG. 4B is a schematic illustration of a tick remover in accordance with another non-limiting embodiment;

FIG. 5A is an exploded view of a tick remover and packaging in accordance with another non-limiting embodiment of the disclosure;

FIG. 5B is a schematic illustration of a tick remover sealed in packaging in accordance with the embodiment of FIG. 5A;

FIG. 5C is a schematic illustration of packaging to hold a removed tick in accordance with another non-limiting embodiment of FIG. 5A;

FIG. 5D is a schematic illustration of packaging sealed with a tick inside.

FIG. 6A is a partially assembled view of a tick remover packaging without the tick remover in accordance with another non-limiting embodiment;

FIG. 6B is a schematic illustration of a tick remover sealed in packaging in accordance with another non-limiting embodiment;

FIG. 6C is a schematic illustration of a tick remover partially in packaging in accordance with another non-limiting embodiment;

FIG. 6D is a schematic illustration of a tick remover packaging in accordance with another non-limiting embodiment; and

FIG. 6E is a schematic illustration of packaging sealed with a tick inside.

### DETAILED DESCRIPTION

Embodiments disclosed herein provide for a tick remover tool that is effective (removing both the tick and the head and barb quickly and effectively), compact/portable (thin (substantially flat)/fits in a wallet), sterile and individually wrapped (does not further infect the area when removing the tick head), and is low cost. In some embodiments the packaging can be used to contain the tick. A sharp tip can be included on the tool, and the packaging can protect the user from the sharp tip while removing the body of the tick. In some embodiments there is a cover or shield around the sharp point used to remove the tick head, again to protect the user from inadvertent injury from the sharp point during removal of a tick.

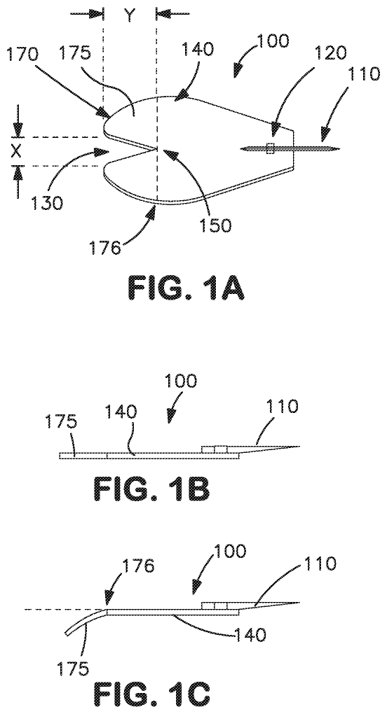

Referring to FIG. 1A, an embodiment of a tick remover 100 is illustrated, wherein the tick remover 100 is a plate 140 with a remover 110 on one surface or edge and a notch 130 on another surface or edge. In another embodiment, plate 140 is substantially flat and is made of metal such as stainless steel, spring steel, plated steel, zinc, aluminum, etc. This can be manufactured using a progressive stamping die, die casting, etc. In another embodiment plate 140 can be made of plastic, such as ABS, POM, PA (nylon), PP, etc. and can be manufactured using injection molding, 3D printing, or machining. In one embodiment the thickness of the plate 140 is between 0.05 and 1.25 mm. The overall length of the tick remover 100 is 2 and 7 cm and the width is between 0.5 and 3 cm. In one embodiment the length is less the 5 cm and the width is between 0.25 and 1 cm.

The size and dimensions of plate 140 as mentioned above are useful in providing a tool which is easy to store and transport, and which is also form fitting to the hand of a user for effective use when needed, especially compared to a needle that has a very small diameter and is difficult to hold.

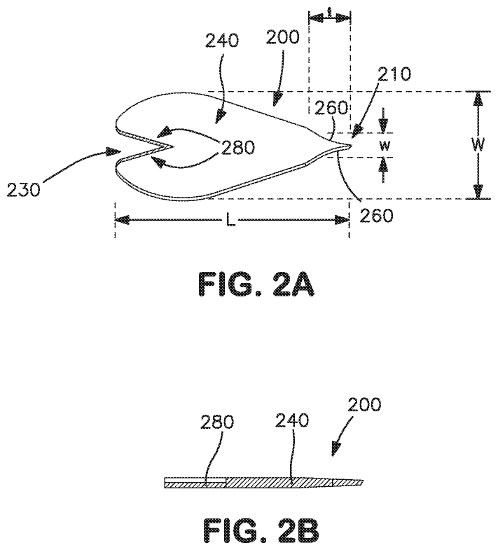

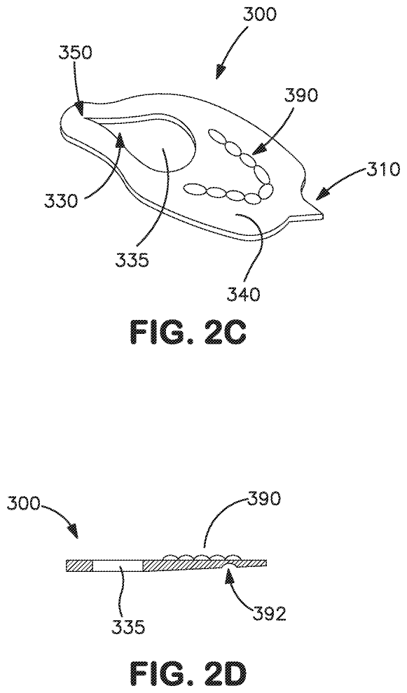

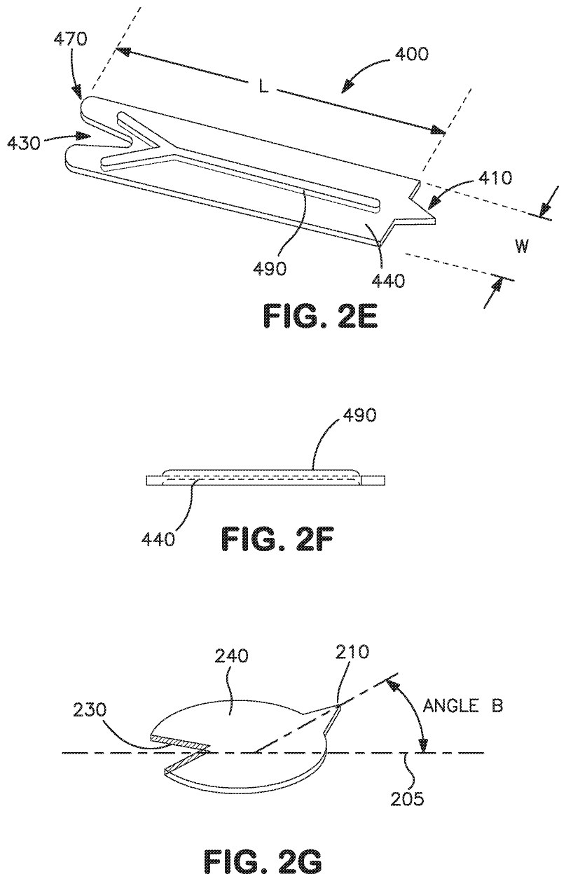

The remover 110 can be a sharp point or needle structure which extends from plate 140 for use when needed. In FIG. 1A, remover 110 is structured as a needle, that is, as an elongate object having a sharp point. In other embodiments such as that illustrated in FIG. 2A and further discussed below, the remover (210 FIG. 2A) can also have sharp sides or edges which can also help in removing any remaining objects after removal of the tick. In another embodiment the remover 110 can be fixed or removable by the end user.

Still referring to FIG. 1A, remover 110 can be fastened to plate 140 via welding, crimping, ultrasonic welding, etc. schematically shown at 120 in FIG. 1A. In another embodiment, such as is shown in FIG. 2A, the remover 110 can be integral to plate 140. In another embodiment the remover 110 is removable. Remover 110/210 has at least one sharp edge to remove the skin around the tick head. In one embodiment, remover 110/210 has a sharp point to help remove the tick head. As mentioned above, remover 110 can be shaped like a needle or pin with a sharp point and a rounded/cylindrical shape. In one embodiment, remover 110/210 can be stamped metal, with a sharp point. One advantage of stamped metal is that a remover such as remover 210 (FIG. 2A) can be provided with side edges 260 that can also be sharp to easily cut or remove skin around the tick head to make it easier to remove. In either case, since the remover 110/210 will cut the skin when needed to remove a tick head, it is appropriate for remover 110/210 and the entire tick remover 100/200 to be sterilized.

Notch 130 is defined at the primary end of the tick remover, and can be used for removing a tick. Notch 130 narrows down to an end 150 which defines the end of notch 130 and the typical area where a tick removal will take place. In one embodiment, end 150 is defined as a small radius and in another embodiment can be a sharp corner. The notch 130 is defined between arms 175 which in this embodiment are designed with rounded ends 170. In another embodiment, arms 175 can be flat as shown in FIG. 1B. In one embodiment arms 175 can be angled or radiused down or up to define a pivot 176 as shown in FIG. 1C. This can help facilitate the ability to slide the arms 175 under the tick and then use the pivot 176 as a fulcrum to remove the tick. FIG. 1C shows an angle A between the plane of plate 140 and angled arms 175 which can be greater than 0 and less than 90 degrees. In a further embodiment, this angle A can be between 10 and 30 degrees.

Notch 130 in the illustrated embodiment is substantially v-shaped. Inside edges of the arms defining the v-shape can be at an angle which in one embodiment can be between 2 and 60 degrees. In another embodiment the angle can be between 5 and 20 degrees.

In another embodiment inside edges defining the v-shape can be at least partially parallel to define a slot leading up to end 150. Further alternatively, the inside edges can be configured to define a combination of angled and parallel surfaces along the length of the substantially v-shaped notch. Notch 130 can have an opening x between 1 and 10 mm. In another embodiment dimension x is between 1 and 5 mm. Notch 130 can have a depth y between 2 and 12 mm. In another embodiment the depth of the notch is between 5 and 10 mm in length.

Notch 130 and remover 110 are both positioned on surfaces of the remover. These surfaces can be edges of plate 140, and notch 130 can be positioned sufficiently distant from remover 110 that both structures can be used as desired without interfering with each other. In one configuration, the remover 110 (or 210 and others that follow in the discussion below) can be located on the side opposite notch 130. In another embodiment the sharp tip/remover 110/201 can be located on a surface or edge of the tick remover 100/200 which is not opposite, but can be at a different angle around the body of the tick remover, for example extending off to a side at an angle of about 90 degrees from the notch.

Referring to FIG. 2A, tick remover 200 is similar to tick remover 100 except the remover 210 is integral to the plate 240 to reduce the cost of production. Further, in this configuration remover 210 can have sides 260 which can be sharp to help in removing any remaining foreign objects after removal of the tick.

In one embodiment notch 230 can have at least one beveled edge 280 to help facilitate sliding the tick remover 200 under the tick as far back into the notch 230 as possible such that the entire tick is removed. FIG. 2B illustrates tick remover 210 in cross section to further illustrate beveled edge 280.

Referring back to FIG. 2A, the tick remover of this disclosure can have a length L and width W which are selected to facilitate both ease of use and transportability. Further, the remover component 210 can also have a length 1 and width w, all as shown in FIG. 2A. The width W of tick remover 200 can be between 0.25 and 2.5 cm such that it is easy to hold. The length L of tick remover 200 can be between 1 and 8 cm. The length 1 of remover 210 can be between 1 mm and 8 mm. In another embodiment the length 1 is between 2 mm and 6 mm. The width w of the remover 210 can be between 0.5 mm and 4 mm. In another embodiment the width w can be between 1 mm and 3 mm.

Referring to FIG. 2C, a tick remover 300 is illustrated which is similar to tick removers 100 and 200, except the notch 330 is reversed within an opening 335. The opening 335 is large enough to place over the tick and then slide notch 330 towards the tick, until the tick is positioned in the smallest part of the end 350 that is possible prior to lifting or pivoting tick remover 300 to remove the tick. Tick remover 300 in this embodiment also contains ribs 390 formed or defined on plate 340. The ribs 390 are raised material to increase the strength of plate 340 of the tick remover 300. Ribs 390 allow the use of thinner material while maintaining the required stiffness of plate 340. In one embodiment, the height of the ribs is 50% to 1000% (10 times) the thickness of plate 340. FIG. 2D shows a cross section of the tick remover 300 with interrupted ribs 390. Ribs 390 can be made from deformed material 392 of the plate 340 to the desired height. In another embodiment the ribs 390 can be added to the plate 340. The ribs can be interrupted as shown in FIG. 2D. In another embodiment the ribs 390 can be continuous as shown in FIG. 2E.

Referring to FIG. 2E, tick remover 400 is similar to other tick removers disclosed herein (such as tick removers 100 and 200) with an integrated remover 410 and continuous ribs 490. In addition, the width W and length L of tick remover 400 is minimized for portability. The width W can be between 0.25 and 2 cm. In one embodiment the length L can be between 2 and 7 cm. In another embodiment the length is less than 5 cm.

In another embodiment, portability is further improved by minimizing the weight of the tick remover 400 and packaging to less than 50 grams. In another embodiment the weight of the tick remover and packaging is less than 5 grams. In another embodiment the weight of the tick remover and packaging is less than 2 grams. In one embodiment the weight of the tick remover alone can be less than 10 grams. In another embodiment the tick remover is less than 2 grams. In another embodiment the tick remover is less than 0.5 grams.

In another embodiment tick remover 400 has an overall thickness of less than 1.25 mm. In another embodiment the tick remover and packaging has an overall thickness of less than 0.5 mm. In one embodiment, the width of the tick remover with packaging is 5 cm or less, and can be between 0.5 and 5 cm. The length of the tick remover with packaging can be between 3 and 9 cm. In one embodiment, the length of the tick remover with packaging is less than 9 cm. In one embodiment, the thickness of tick remover and packaging is less than 5 mm. In another embodiment the thickness of the tick remover and packaging is less than 1.5 mm. The weight and size of the product and packaging allow for extremely easy transportation as it takes up minimal volume and weight such that it would not be noticeable to the person carrying the product. For example, with the width of 4 cm and 8 cm in length and the thickness of 0.1 cm the volume is approximately 3.2 cm³ and the weight of approximately 1.1 grams which is approximate the same volume as a US dime, and half the weight. The product and packaging is much smaller in size and thin that it has less volume than a credit card.

In another embodiment the volume of the tick remover is less than 5 cm³. In one embodiment the volume of the tick remover is less than 0.5 cm³. In another embodiment the volume of the tick remover is less than 0.1 cm³.

In another embodiment the volume of the tick remover and packaging is less than 60 cm³. In one embodiment the volume of the tick remover and packaging is less than 20 cm³. In another embodiment the volume of the tick remover and packaging is less than 2 cm³.

FIG. 2F shows a side view of tick remover 400 with ribs 490 to provide the desired stiffness to the tick remover tool.

FIG. 2G shows an alternate version of a tick remover similar to the embodiment illustrated in FIG. 2A, but with remover 210 positioned at an angle B to a center line 205 of body 240 such that remover 210 is on an edge of body 240 but not diametrically opposite to notch 230. This helps to allow holding and manipulation of tick remover 200 from the end opposite to notch 230 without sharp remover 210 being right where a user might want to hold the device.

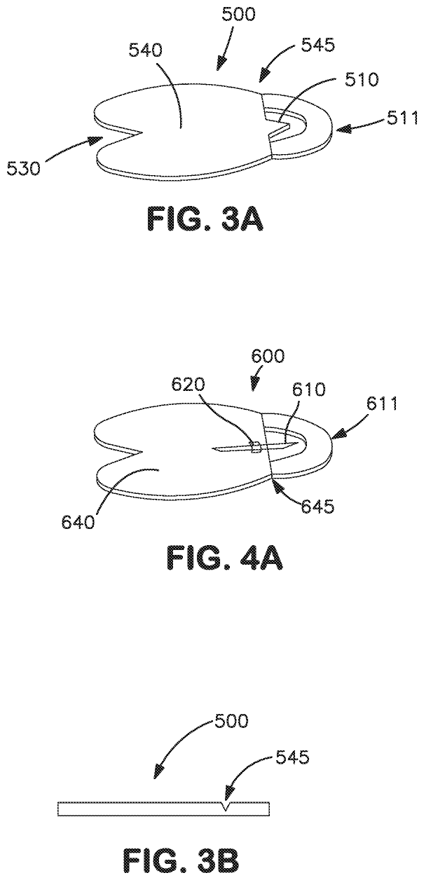

Referring to FIG. 3A, tick remover 500 includes a cover or shield 511 which extends around and beyond remover 510. The shield 511 is to protect an end user from contacting the sharp point or edges of the remover 510 when using the notch 530 to remove the tick. At the connection between shield 511 and plate 540 an indent 545 can be provided. Indent 545 can be made using known manufacturing techniques such as by depressing the material in a stamping die or having the protrusion in an injection mold. In one embodiment, the indent 545 has a sharp corner that makes it easier to remove the shield 511 when desired. Once the tick is removed, shield 511 can be removed by bending the shield 511 up and down in relation to plate 540 until the shield comes off, or simply bending shield 511 over once to be flat with plate 540. FIG. 3B shows a side view of the indent 545.

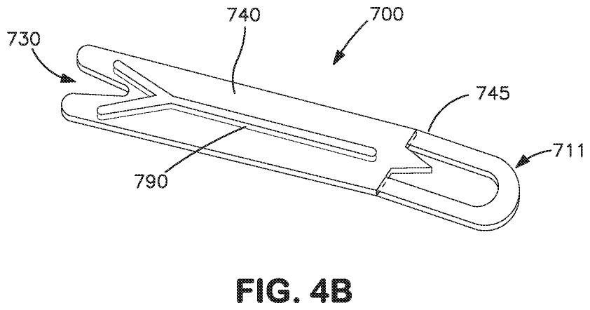

Referring to FIG. 4A, a tick remover 600 is shown which combines features of tick remover 100 and 500, adding the shield 611 onto plate 640 with indent 645.

Referring to FIG. 4B, tick remover 700 is shown which combines features of tick remover 400 and 500, adding a shield 711 onto plate 740 with ribs 790 and indent 745.

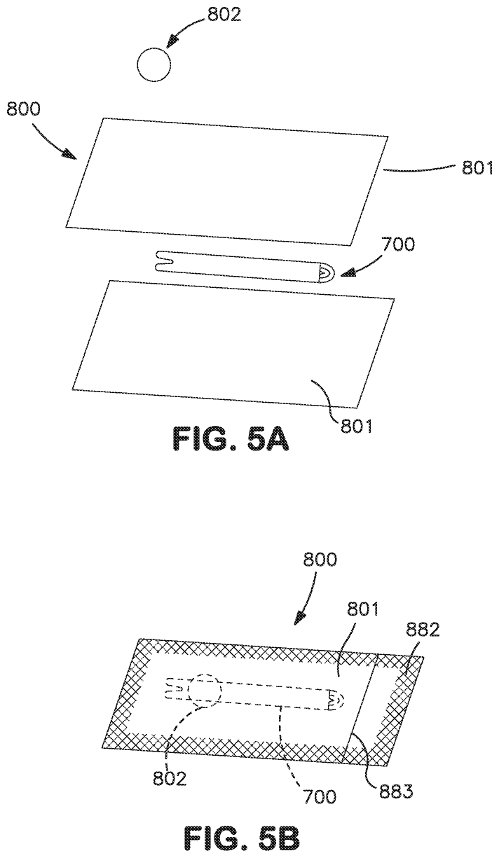

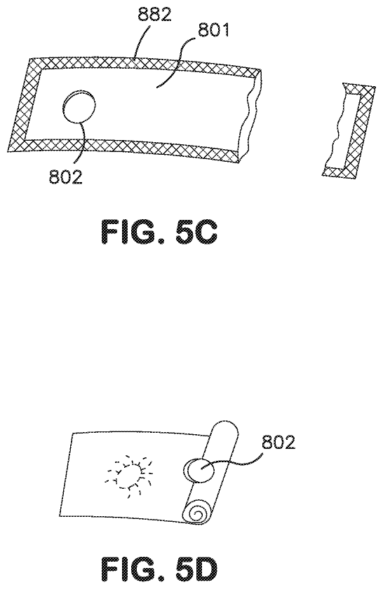

FIGS. 5A and 5B show packaging 800 to hold the tick remover 700. Alternatively this feature could be implemented with the embodiments of tick remover 100, 200, 300, 400, 500, or 600. The packaging can be made from paper or thin sheets of plastic or multi-layer packaging material, for example in two sheets 801 to enclose the tick remover, and sealed 882 on at least one side. Sealing can be performed using known processes such as heat sealing or ultrasonic or similar processes. This keeps the tick remover clean and sterilized as desired. In one embodiment, a tear line location 883 can be printed on the packaging. Tear line 883 is preferably located near one edge of packaging 800. In one embodiment a notch can be located at the edge of the paper or plastic to facilitate tearing the paper or plastic sheet as known in the art. In one embodiment, reusable sticker 802 is placed on the outside package 800, on one sheet 801 of the paper. Sticker 802 can be reusable with an adhesive similar to the adhesive used on 3M POST-IT notes. The function of reusable sticker 802 is set forth below.

FIG. 5C shows opening of packaging 800 by tearing at the tear line 883. The tool has been removed for use. Once the tick is removed, the tick can be inserted into package 800 and an edge of the package can be rolled or folded and held closed with sticker 802, see FIG. 5D. This prevents the tick from escaping or falling out so that it can be saved for testing as needed. It should be appreciated that while FIGS. 5B and 5C show a sticker 802 for use in sealing the package with a tick inside, any suitable fastener could be used for this purpose, including but not limited to paper clips, staples or the like. Different types of fasteners may be preferred for different configurations, for example where no adhesives are desired. In addition, instructions or guidance markings could be provided to guide a user to fold the packaging, without a fastener, to secure the removed tick.

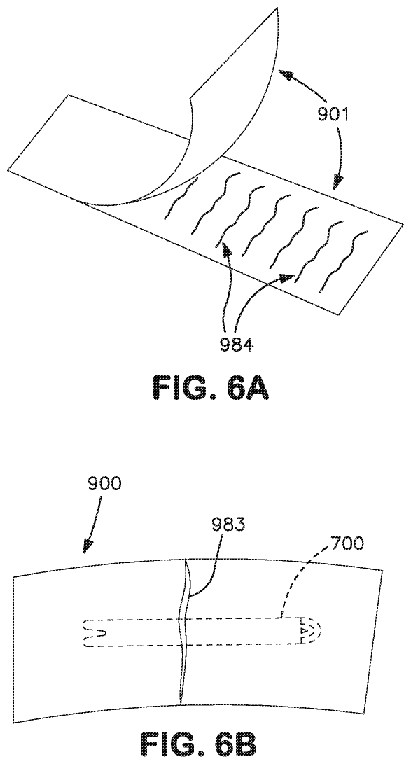

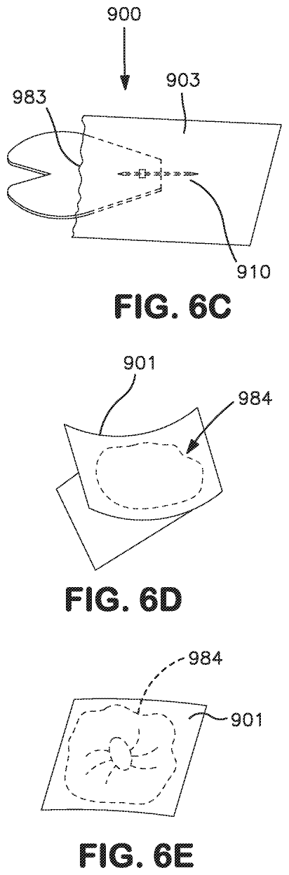

FIGS. 6A-6D show a packaging 900. Packaging 900 is made of paper or plastic sheets or multilayer materials used in packaging 901 and glue or adhesive 984. The adhesive 984 can also be similar to the adhesive used on 3M POST-IT notes such that the packaging 900 keeps the tick remover (100, 200, 300, 400, 500, 600, or 700), sealed and sterile if desired, and can be separated to remove the tick remover. In one embodiment, packaging 900 can have a tear line 983 printed on the surface of paper 901. In one embodiment a notch can be located at the edge of the paper or plastic to facilitate tearing the paper or plastic sheet as known in the art. As shown in FIG. 6C packaging covering the remover 910 can protect the end user from contacting the sharp point of the remover while removing the tick. Specifically, by tearing at tear line 983, a portion 903 of packaging 900 can be left in place over remover 910. In addition, packaging removed from the notch side can be used to contain the tick between the adhesive 984 as shown in FIGS. 6D and 6E. Once the tick is removed from the skin it can be placed on the adhesive 984 and then the top layer of paper with adhesive 984 can be pressed to seal in the tick. In one embodiment only one of the papers 901 has adhesive. In another embodiment both papers 901 have adhesive. This contains the tick such that it can be sent for testing if desired or if any symptoms appear.

It should be appreciated that while the various embodiments of remover 110, 210, etc., pair well with the tick remover aspect of the present disclosure, this remover can also be used to remove other debris from the skin which may or may not be related to a tick bite, and could be any type of scrap or foreign matter embedded in the skin. This added functionality can be beneficial, but is particularly well suited when combined with the tick remover aspect of the articles of the present disclosure.

It should be appreciated that the various embodiments disclosed herein provide an advantageous tick removing tool which addresses numerous issues identified in the background discussion above. It should also be appreciated that the various embodiments shown in the attached drawings and described herein have features which could be combined with other features from different embodiments, without specific restriction of any particular combination of disclosed features.

The term “about” is intended to include the degree of error associated with measurement of the particular quantity based upon the equipment available at the time of filing the application.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the disclosure is provided in detail in connection with only a limited number of embodiments, it should be readily understood that the disclosure is not limited to such disclosed embodiments. Rather, the disclosure can be modified to incorporate any number of variations, alterations, substitutions or equivalent arrangements not heretofore described, but which are commensurate with the spirit and scope of the disclosure. Additionally, while various embodiments of the disclosure have been described, it is to be understood that the exemplary embodiment(s) may include only some of the described exemplary aspects. Accordingly, the disclosure is not to be seen as limited by the foregoing description, but is only limited by the scope of the appended claims.

What is claimed is:

1. A tick remover device, comprising:
a substantially flat body defining a substantially v-shaped notch on one surface or edge of the flat body;
a sharp point on another surface of the flat body; and
packaging material enclosing the flat body.

2. A tick remover device, comprising:
a substantially flat body defining a substantially v-shaped notch on one surface or edge of the flat body;
a sharp point on another surface of the flat body; and
a protective means covering the sharp point to protect a user from the sharp point during use of the v-shaped notch to remove a tick.

3. A packaged tick remover, comprising:
a package containing the tick remover device according to claim 1, wherein the tick remover device is sterilized.

4. A packaged tick remover, comprising:
a package containing the tick remover device according to claim 1, wherein the package is made of paper.

5. A packaged tick remover, comprising:
a package containing the tick remover device according to claim 1, wherein at least one interior surface of the package has adhesive to capture the tick and/or hold the package in place while covering the sharp point.

6. A packaged tick remover, comprising:
a package containing the tick remover device according to claim 1, further comprising a fastener to secure a tick in the package.

7. A packaged tick remover, comprising:
a package containing the tick remover device according to claim 1, wherein the package is free of adhesive.

8. The tick remover device of claim 1, wherein the substantially flat body is a substantially flat metal plate, and wherein the sharp point is a needle attached to the substantially flat body or formed integrally with the body.

9. The tick remover device of claim 2, wherein the protective means comprises a breakaway shield which can be removed from the substantially flat body to expose the sharp point.

10. The tick remover device of claim 1, wherein the notch has an angle of between 2 and 60 degrees.

11. The tick remover device of claim 1, wherein the notch defines an opening having a width x between 1 and 10 mm and a length y between 2 and 12 mm.

12. The tick remover device of claim 1, wherein the v-shaped notch is defined by inner edges of arms of the plate member, and wherein at least a portion of the inner edges are parallel.

13. The tick remover device of claim 1, wherein the tick remover has a width of between 0.25 and 2.5 cm, a length of between 1 and 8 cm, a thickness of less than 2 mm, a weight of less than 10 g, and a volume less than 5.0 cm³.

14. The packaged tick remover of claim 3, wherein the packaged tick remover has a width between 0.5 and 5 cm, a length of between 3 and 9 cm, a thickness of between 1 and 5 mm, a weight of less than 50 g, and a volume of less than 60 cm³.

15. The tick remover device of claim 1, wherein sides of the V-shaped notch are beveled.

16. The tick remover device of claim 1, wherein the substantially flat body comprises arms which define the V-shaped notch, and wherein the arms are co-planar with the substantially flat body.

17. The tick remover device of claim 1, wherein the substantially flat body comprises arms which define the V-shaped notch, and wherein the arms are bent at an angle greater than 0 and less than 90 degrees with respect to the body.

18. The tick remover device of claim 1, further comprising stiffening material positioned along the substantially flat body, wherein the stiffening material comprises at least one rib formed on the body.

19. The packaged tick remover of claim 3, wherein the packaging material covers both sides of the tick remover device, and wherein a portion of the packaging material can be removed away from a notch end of the tick remover device to expose the notch end for use, while remaining in place over the sharp point.

20. A method for removing a tick from the skin, comprising the steps of:
removing a tick remover device from a packaging enclosing the tick remover device, wherein the tick remover device comprises a substantially flat body defining a substantially v-shaped notch on one surface; a sharp point on another surface; and packaging material enclosing the flat body;
placing the notch against a tick embedded in the skin to remove the tick;
using the sharp point to remove any remaining portions of the tick; and
sealing the removed tick into the packaging.
